(19) United States
(12) Patent Application Publication
(10) Pub. No.: US 2021/0290932 A1
(43) Pub. Date: Sep. 23, 2021

(54) PUMP HOUSING DEVICE, METHOD FOR PRODUCING A PUMP HOUSING DEVICE, AND PUMP HAVING A PUMP HOUSING DEVICE

(71) Applicant: KARDION GMBH, Stuttgart (DE)

(72) Inventor: Ingo Stotz, Ditzingen (DE)

(21) Appl. No.: 17/057,548

(22) PCT Filed: May 30, 2019
(86) PCT No.: PCT/EP2019/064141

§ 371 Date: May 19, 2021

(30) Foreign Application Priority Data
May 30, 2018 (DE) 10 2018 208 540.4

(51) Int. Cl. A61M 60/135

(52) U.S. Cl. CPC A61M 60/135; A61M 2207/00

(57) ABSTRACT

The approach presented here relates to a pump housing device (300) for guiding a fluid (215). The pump housing device (300) has a housing apparatus (115) and a diffuser (305). The housing apparatus (115) is designed to accommodate at least one component of a pump (105) for pumping the fluid (215) through the housing apparatus (115), the housing apparatus (115) having at least one outlet opening (120) for letting out the fluid (215) which can be or is pumped by the pump (105). The diffuser (305) is or can be coupled to the housing apparatus (115) and/or to a supply hose is arranged and/or formed in an operating position (310) in order to guide the pumped fluid (215) transversely to the outlet opening (120) after said fluid has passed through the outlet opening (120).

The approach proceeds from a device or a method according to the class of the independent claims.

For cardiovascular support of patients having heart failure, heart support systems, so-called VAD (ventricular assist device) systems, are used, which take over part or all of the pumping function for a blood flow of the heart. These systems are subdivided into temporary systems for short-term heart support and permanent systems. The temporary systems are inter alia used to bridge the time until a suitable donor heart is available and can be implanted. The permanent systems are used for long-term retention on or in the patient. One component of such a system is a blood pump, typically a centrifugal pump in the form of a turbo pump, which is driven by an integrated electric motor and produces the required blood flow by means of an impeller. The pump can be implanted at different locations. The pump can be sutured to the heart from the outside by means of an invasive surgery, a sternotomy, or it is placed transfemorally or transaortally into the aorta or completely or only partially into the ventricle in a minimally invasive manner by means of a catheter.

Proceeding therefrom, the invention is based on the object of specifying an improved pump housing device and pump, in particular for a heart support system, in order to achieve a high efficiency, and a suitable method for its production.

Against this background, with the approach presented here, a pump housing device for guiding a fluid, furthermore a method for guiding a fluid, and lastly, a pump with a pump housing device according to the main claims are presented. Advantageous developments and improvements of the pump housing device specified in the independent claim are possible by means of the measures listed in the dependent claims.

The advantages achievable with the presented approach consist in that a pressure loss of a pump operated in connection with the pump housing device presented here is very low. Pump efficiency is therefore advantageously high.

A pump housing device for guiding a fluid has a housing apparatus and a diffuser. The housing apparatus is formed to accommodate at least one component of a pump for conveying the fluid through the housing apparatus, wherein the housing apparatus has at least one outlet opening for letting out the fluid that can be or is conveyed by the pump. The diffuser is or can be coupled with the housing apparatus and/or an inlet hose and, in an operating position, is arranged and/or formed to guide the conveyed fluid transversely to the outlet opening after said fluid has passed through the outlet opening.

The inlet hose can be formed to be coupled or couplable with the housing apparatus. For example, one end of the inlet hose can be fluidically coupled or couplable with a start of the housing apparatus. At least one component of the diffuser can be formed in one piece with the inlet hose.

The outlet opening can extend through a circumference of the housing apparatus, which can be cylindrical or tubular, for example. A flow direction of the fluid through the housing apparatus can be arranged in at least one component transversely, for example orthogonally, to the outlet opening. The flow direction in at least one component can be parallel to a longitudinal axis of the pump that is or can be accommodated in the housing apparatus.

A pump housing device presented here allows a fluid to be guided after said fluid has passed through an outlet opening, which can be arranged transversely to a desired flow direction in order to reduce an impending pressure loss of the fluid upon passing into an outer region arranged outside the housing apparatus. This outer region can, for example, have a larger diameter than the housing apparatus arranged therein.

The diffuser can be formed to be transferable from a rest position to the operating position and/or from the operating position to the rest position, wherein the diffuser can in particular be formed so that it can be folded out from the rest position to the operating position. In the rest position, the diffuser can be arranged in a folded-in state on the housing apparatus, e.g., be abutting thereon. In the rest position, the pump housing device can thus be easily introduced or inserted into the outer region. For example, the diffuser can in this case be designed to be transferable from the rest position to the operating position by a temperature, e.g., by increasing a temperature.

The diffuser can be arranged or arrangeable circumferentially around the housing apparatus. An arrangement of a cylindrical or tubular housing apparatus in an outer region which is, for example, likewise cylindrical or tubular is thus made possible. If the housing apparatus according to an advantageous embodiment has a plurality of outlet openings, which can, for example, be arranged circumferentially around the circumference of the housing apparatus, the fluid can be guided circumferentially around the housing apparatus after said fluid has passed through the outlet openings. This allows a very large volume flow of fluid through the pump housing device into the outer region.

In the operating position, a lateral surface of the diffuser or the diffuser itself can have a cross-sectional area that increases in the flow direction of the fluid. This allows the fluid to be guided with a very low pressure loss. For example, in the operating position, the diffuser can have the shape of a truncated cone or a dome or an umbrella.

The diffuser can have a support structure, in particular with at least one strut, and/or a flexible jacket. Circumferentially, the diffuser can also have a plurality of struts, e.g., arranged radially. For example, the struts can have a material, such as Nitinol, that has a shape memory. This facilitates a transfer from the rest position to the operating position, wherein the transfer can be brought about, for example, by the temperature. The flexible jacket can have silicone and/or polyurethane, “PU” for short.

A pump with one of the pump housing devices presented above is also presented. At least one component of the pump is accommodated in the housing apparatus, wherein the pump is in particular formed as an axial pump. The pump can have an axially flowable impeller for conveying the fluid in the flow direction. A pump of this type with the pump housing device is formed for use in a tubular outer region.

Such a pump with the pump housing device can particularly advantageously be formed as part of a heart support system to be accommodated in a blood vessel. The blood vessel may be an aorta.

Lastly, a method for producing a pump housing device is presented. The method comprises a step of coupling a diffuser with the housing apparatus, wherein the diffuser can be transferred into an operating position in order to guide the conveyed fluid transversely to the outlet opening after said fluid has passed through the outlet opening.

This method can, for example, be implemented in software or hardware or in a mixed form of software and hardware in a control device, for example.

Exemplary embodiments of the approach presented here are shown schematically in the drawings and explained in more detail in the following description. They show:

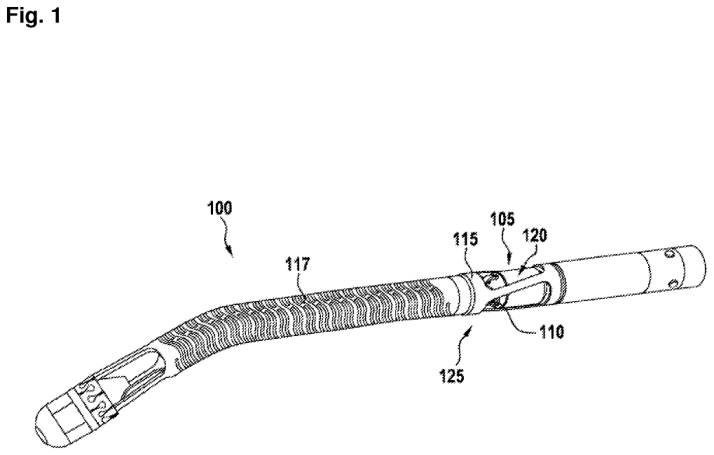

FIG. 1 a perspective side illustration of a pump device for use with a pump housing device in a blood vessel;

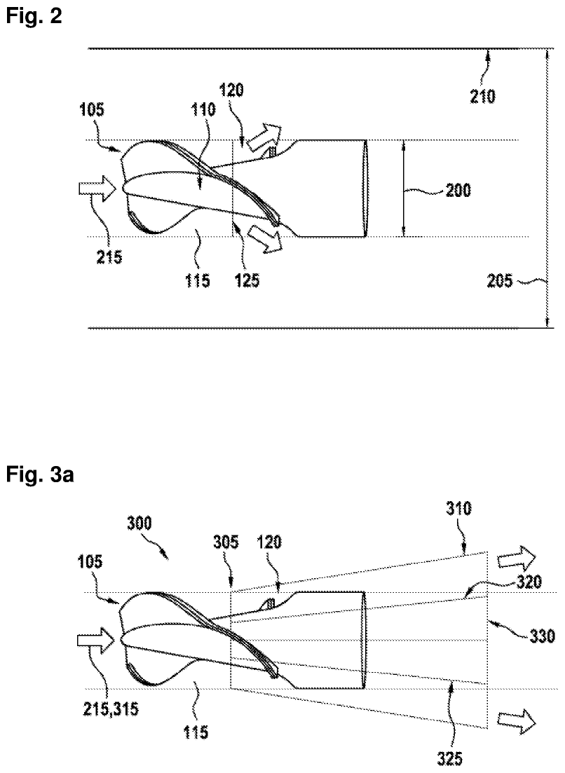

FIG. 2 a schematic side view of a pump for use with a pump housing device;

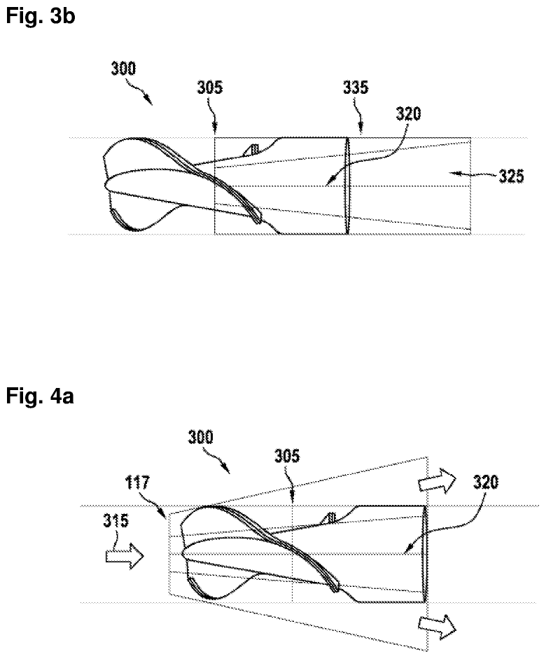

FIG. 3a a schematic side illustration of a pump housing device for guiding a fluid;

FIG. 3b a schematic side illustration of a pump housing device according to an exemplary embodiment;

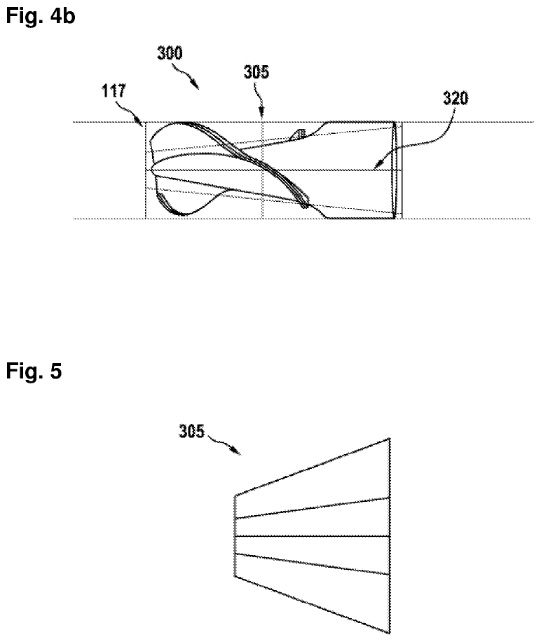

FIG. 4a a schematic side illustration of a pump housing device according to an exemplary embodiment;

FIG. 4b a schematic side illustration of a pump housing device according to an exemplary embodiment;

FIGS. 5-8 various embodiments of a diffuser of a pump housing device in a side view;

FIG. 9 a flow diagram of a method for producing a pump housing device.

In the following description of favorable exemplary embodiments of the present approach, the same or similar reference signs are used for the elements that are shown in the various figures and have a similar effect, wherein a repeated description of these elements is omitted.

If an exemplary embodiment includes an “and/or” conjunction between a first feature and a second feature, this should be read to mean that the exemplary embodiment according to one embodiment comprises both the first feature and the second feature and according to another embodiment comprises either only the first feature or only the second feature.

FIG. 1 shows a schematic side illustration of a pump device 100 for use with a pump housing device.

The pump device 100 can be arranged in a minimally invasive manner by means of a catheter transfemorally or transaortally in an aorta and/or at least partially in a ventricle. The pump device 100 can therefore also be referred to as a blood pump for a heart support system. A maximum outer diameter of the pump device 100 shown here is limited to less than ten millimeters, which is why the pump device 100 has a pump 105 of axial design, i.e., with an axial-flow impeller 110. During operation of the pump device 100, a fluid to be conveyed, in this case blood, flows through an inlet hose 117 and is ejected through outlet openings 120 attached to a circumference of a housing apparatus 115 of the pump 105 in order to be fed back into the aorta. This is made possible and realized according to FIG. 1 by the impeller 110 in a first section being completely enclosed by the housing apparatus 115 in the form of a cylindrical pump housing and in a second section being located within a housing region of the housing apparatus 115 interrupted by the outlet openings 120. A transition between these two sections is characterized by a beginning 125 of the outlet openings 120, which can also be referred to as discharge openings or housing discharge openings.

FIG. 2 shows a schematic side illustration of a pump 105 for use with a pump housing device. This pump can be the pump 105 with the impeller 110 described with reference to FIG. 1, this pump being arranged in the housing apparatus 115 also described in FIG. 1.

Since a pump outer diameter 200 of the pump 110 is significantly smaller than a vessel diameter 205 of the vessel, here by way of example the aorta 210, into which the fluid 215, here in the form of blood, flows out after said fluid has passed through the outlet openings 120, a large, abrupt cross-sectional jump leads to large permanent total pressure losses and thus to a reduced pump efficiency of the pump 105.

The pump housing device shown in the following figures is designed to prevent such a reduction of the pump efficiency.

FIG. 3 a) shows a schematic side illustration of a pump housing device 300 for guiding a fluid 215 according to an exemplary embodiment. This can be a pump housing device 300 for use with one of the pumps 105, described in FIG. 1 or 2, with an impeller.

The pump housing device 300 is designed to guide the fluid 215. For this purpose, the pump housing device 300 has the housing apparatus 115 described in FIG. 1 or 2, which is formed to accommodate at least one component of the pump 105 for conveying the fluid 215 through the housing apparatus 115. As already described in the previous figures, this housing apparatus 115 has the at least one outlet opening 120 for letting out the fluid 215 conveyable or conveyed by the pump 105. In addition, the pump housing device 300 has a diffuser 305, which is or can be coupled according to this exemplary embodiment with the housing apparatus 115 and, in an operating position 310 shown here, is arranged and/or formed to guide the conveyed fluid 215 transversely to the outlet opening 120 after said fluid has passed through the outlet opening 120.

According to this exemplary embodiment, the diffuser 305 is arranged circumferentially around the housing apparatus 115. In the operating position 310, a lateral surface of the diffuser 305 according to this exemplary embodiment has a cross-sectional area that increases in the flow direction 315 of the fluid 215. Alternatively, according to this exemplary embodiment, the diffuser 305 itself can also have a cross-sectional area that increases in the flow direction 315 of the fluid 215. In this case, the diffuser 305 according to this exemplary embodiment has the shape of a truncated cone in the operating position 310.

Optionally, the diffuser 305 has a support structure with at least one strut 320 and/or a flexible jacket 325. According to this exemplary embodiment, the diffuser 305 has a plurality of these struts 320.

According to this exemplary embodiment, the diffuser 305 is formed to be transferable from a rest position to the operating position 310 and/or from the operating position 310 to the rest position, wherein the diffuser 305 according to this exemplary embodiment is formed so that it can be folded out from the rest position to the operating position 310.

According to this exemplary embodiment, at least one component of the pump 105 according to the description in FIG. 1 or 2 is accommodated in the housing apparatus 115 of the pump housing device 300. According to this exemplary embodiment, the pump 105 is formed as an axial pump.

Already described details of the pump housing device 300 are described once again below in other words:

In combination with the pump 105, the pump housing device 300 presented here can also be referred to as a pump with an unfoldable device for increasing efficiency.

Thanks to the pump housing device 300, the pressure loss of the pump 105 described in FIGS. 1 and 2 can be reduced. By using the pump housing device 300, the efficiency of the pump 105 is thus increased in comparison to an application without a pump housing device 300. This reduction of the pressure loss described above is reduced by introducing a device in the form of the diffuser 305. The device is designed in such a way that the cross-sectional change shown and described in FIG. 2 is less abrupt or, in other words, the cross-sectional jump is less pronounced. Advantages in comparison to an embodiment without diffuser 305 result, in summary, in a better flow routing and lower pressure losses as well as an increased efficiency.

An exemplary description of a sequence during operation of the pump 105 in conjunction with the pump housing device 300 presented here follows:

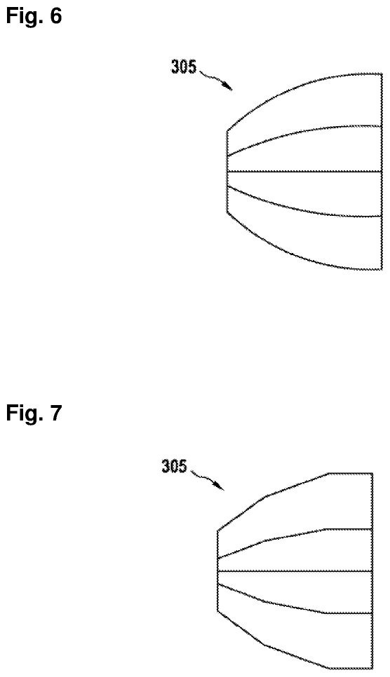

The fluid 215 in the form of blood is fed from the ventricle through a so-called inlet hose to an active pump part, to the impeller among other things. The impeller is enclosed on the outside by a housing apparatus 115, preferably a cylindrical housing apparatus according to this exemplary embodiment, which partially has openings. The pump housing device 300 supplements the pump 105 with the housing apparatus 115 from FIGS. 1 and 2 with an additional device, the diffuser 305, which is permanently or detachably connected to the housing apparatus 115. The diffuser 305 is designed in such a way that it is flexible, crimpable, foldable, and unfoldable according to this exemplary embodiment. This offers the advantage that in the folded or crimped state, it can nestle closely to the housing apparatus 115 and thus allows minimally invasive implantation. According to this exemplary embodiment, the diffuser 305 is designed in such a way that it has a support structure with several struts 320 made of Nitinol as well as a flexible jacket 325, which is completely or at least partially closed in the circumferential direction and is produced according to this exemplary embodiment of silicone and/or PU and/or is permanently or detachably connected to the support structure. Together with the support structure in the unfolded state shown here, the lateral surface serves for flow routing of the fluid 215 in order to reduce losses when said fluid flows out of the outlet opening 120 or the outlet openings 120. The diffuser 305 is characterized in that its lateral surface in the unfolded state encloses a cross-sectional area increasing, i.e., divergent, in the main flow direction 315, i.e., in the direction of the axis of rotation of the impeller. A downstream discharge surface 330 of the diffuser 305 is therefore larger than a connection surface, arranged opposite the discharge surface 330, of the diffuser 305 with the housing apparatus 115. In this case, the diffuser 305 or at least its lateral surface according to this exemplary embodiment is designed in the form of a truncated cone. FIGS. 6 and 7 show further possible designs of an unfolded diffuser 305.

FIG. 3b shows a schematic side illustration of a pump housing device 300 according to an exemplary embodiment. This can be the pump housing device 300 described in FIG. 3a, with the difference that the diffuser 305 according to this exemplary embodiment is arranged in the rest position 335. In the rest position 335, in a folded or crimped state, the diffuser 305 according to one exemplary embodiment is arranged to be nestled closely to the housing apparatus 115 and can thus be minimally invasively implanted.

FIG. 4a shows a schematic side illustration of a pump housing device 300 according to an exemplary embodiment. This can be the pump housing device 300 described in FIG. 3a, with the difference that the diffuser 305 according to this exemplary embodiment is not coupled with the housing apparatus but is arranged at an end of the inlet hose 117. The inlet hose 117 is coupled with the housing apparatus. According to one exemplary embodiment, the inlet hose 117 or at least one section of the inlet hose 117 is part of the pump housing device 300.

A description of a sequence during operation of the pump in conjunction with the pump housing device 300 presented here follows by way of example:

According to this exemplary embodiment, the blood is fed from the ventricle through the inlet hose 117 to the active pump part, at least to the impeller according to this exemplary embodiment. According to this exemplary embodiment, this inlet hose 117 is constructed from a laser-cut, coiled or braided structure, according to this exemplary embodiment made of Nitinol or similar materials with shape memory properties, which according to this exemplary embodiment is additionally encased in a further material, according to this exemplary embodiment silicone and/or polyurethane, in order to produce a closed form. According to this exemplary embodiment, the inlet hose 117 is designed in such a way that at least one or more struts 320 of the diffuser 305 project in the axial direction beyond the outlet opening of the housing apparatus beyond the original end of the inlet hose 117. According to this exemplary embodiment, the struts 320, which can also be referred to as diffuser struts, are part of the inlet hose 117 and thus, like the rest of the inlet hose 117, made of a shape memory alloy. Between the struts 320 is a material, which is used according to this exemplary embodiment for encasing the inlet hose 117, according to one exemplary embodiment silicone and/or PU or according to an alternative exemplary embodiment a material different thereto, which in the unfolded state, i.e., in the operating position shown here, forms a structure closed in the circumferential direction in the form of the diffuser 305. This structure is used for flow routing and reduces the losses during outflow.

FIG. 4b shows a schematic side illustration of a pump housing device 300 according to an exemplary embodiment. This can be the pump housing device 300 described in FIG. 4a, with the difference that the diffuser 305 according to this exemplary embodiment is arranged in the folded-in or crimped state described in FIG. 3b, i.e., in the rest position.

FIG. 5 shows a schematic side view of a diffuser 305 of a pump housing device according to an exemplary embodiment. This can be the diffuser 305 described in FIG. 3a, 3b, 4a, or 4b, which in the operating position has the shape of a truncated cone.

FIG. 6 shows a schematic side view of a diffuser 305 of a pump housing device according to an exemplary embodiment. This can be the diffuser 305 described in FIG. 3a, 3b, 4a, or 4b, which in the operating position has however the shape of a dome according to this exemplary embodiment.

FIG. 7 shows a schematic side view of a diffuser 305 of a pump housing device according to an exemplary embodiment. This can be the diffuser 305 described in FIG. 3a, 3b, 4a, or 4b, which in the operating position has however the shape of an umbrella according to this exemplary embodiment.

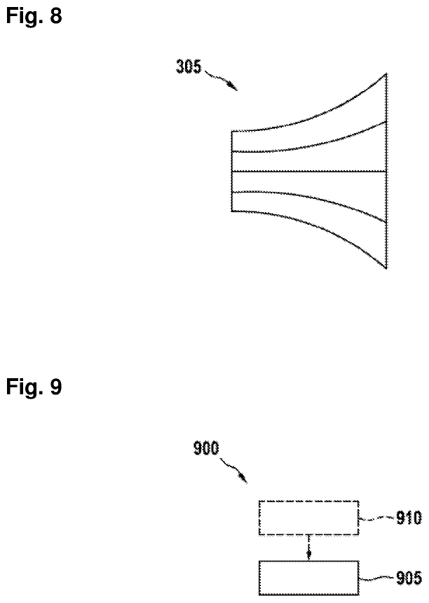

FIG. 8 shows a schematic side view of a diffuser 305 of a pump housing device according to an exemplary embodiment. This can be the diffuser 305 described in FIG. 3a, 3b, 4a, or 4b, which in the operating position has however the shape of a funnel according to this exemplary embodiment. Here, the funnel can basically have an inverted bell shape; the diameter increase can then, for example, increase not linearly as in the case of the truncated cone but with the running length.

FIG. 9 shows a flow diagram of a method 900 for producing a pump housing device according to an exemplary embodiment. This can be a method 900, with the aid of which one of the pump housing devices described with reference to FIGS. 3a to 8 can be produced, which in turn can contain a pump accommodated at least partially therein. The method 900 comprises a step 905 of coupling a diffuser with the housing apparatus, wherein the diffuser can be transferred into an operating position in order to guide the conveyed fluid transversely to the outlet opening after said fluid has passed through the outlet opening.

The method 900 according to this exemplary embodiment optionally moreover comprises a step 910 of mounting, in which the pump housing device is mounted to the pump.

The method steps presented here can be repeated as well as carried out in a sequence other than the one described.

1. A cardiac pump housing device for guiding blood in a blood vessel, the device comprising:
a housing apparatus configured to accommodate at least one component of a pump for conveying the blood through the housing apparatus, wherein the housing apparatus comprises at least one outlet opening configured to facilitate outflow of the blood conveyed by the pump; and
a diffuser which is or can be configured to couple with the housing apparatus and/or an inlet hose, wherein, in an operating position, the diffuser is configured to guide the conveyed blood transversely to the outlet opening after the conveyed blood has passed through the outlet opening.

2-9. (canceled)

10. The pump housing device according to claim 1, wherein the diffuser is configured to be transferable from a rest position to the operating position and/or from the operating position to the rest position, and wherein the diffuser is configured to be unfolded from the rest position to the operating position.

11. The pump housing device according to claim 1, wherein the diffuser is configured to be arranged circumferentially around the housing apparatus.

12. The pump housing device according to claim 1, wherein the diffuser in the operating position has the shape of a truncated cone, a dome, an umbrella, or a funnel.

13. The pump housing device according to claim 1, wherein the diffuser in the operating position has a cross-sectional area that increases in a flow direction of the blood.

14. The pump housing device according claim 1, wherein the diffuser comprises a support structure comprising at least one strut and/or a flexible jacket.

15. A cardiac pump system comprising:
an axial flow pump; and
a pump housing device configured to guide pumped blood, the pump housing device comprising:
a housing apparatus accommodating at least one component of the pump for conveying the blood through the housing apparatus, wherein the housing apparatus comprises at least one outlet opening configured to facilitate outflow of the blood conveyed by the pump; and
a diffuser configured to couple with the housing apparatus and/or an inlet hose, wherein, in an operating position, the diffuser is configured to guide the conveyed blood transversely to the outlet opening after the conveyed blood has passed through the outlet opening.

16. The cardiac pump system according to claim 15, wherein the pump and pump housing device are configured to form part of a heart support system for conveying blood, wherein the pump housing device and diffuser are configured to be delivered to the heart via a blood vessel.

17. The cardiac pump system according to claim 15, wherein the diffuser is configured to be transferable from a rest position to the operating position and/or from the operating position to the rest position, and wherein the diffuser is configured to be unfolded from the rest position to the operating position.

18. The cardiac pump system according to claim 15, wherein the diffuser is configured to be arranged circumferentially around the housing apparatus.

19. The cardiac pump system according to claim 15, wherein the diffuser in the operating position has the shape of a truncated cone, a dome, an umbrella, or a funnel.

20. The cardiac pump system according to claim 15, wherein the diffuser in the operating position has a cross-sectional area that increases in a flow direction of the blood.

21. The cardiac pump system according to claim 15, wherein the diffuser comprises a support structure comprising at least one strut and/or a flexible jacket.

22. A method for producing a cardiac pump housing device, the method comprising:
coupling a diffuser with a housing apparatus,
wherein the housing apparatus is configured to accommodate at least one component of a pump configured to convey blood through the housing apparatus, wherein the housing apparatus comprises at least one outlet opening configured to facilitate outflow of the blood conveyed by the pump, and
wherein the diffuser is configured to transfer into an operating position so as to guide the conveyed blood transversely to the outlet opening after the conveyed blood has passed through the outlet opening.

23. The method according to claim 22, wherein the diffuser is configured to be transferable from a rest position to the operating position and/or from the operating position to the rest position, and wherein the diffuser is configured to be unfolded from the rest position to the operating position.

24. The method according to claim 22, wherein the diffuser is configured to be arranged circumferentially around the housing apparatus.

25. The method according to claim 22, wherein the diffuser in the operating position has the shape of a truncated cone, a dome, an umbrella, or a funnel.

26. The method according to claim 22, wherein the diffuser in the operating position has a cross-sectional area that increases in a flow direction of the blood.

27. The method according to claim 22, wherein the diffuser comprises a support structure comprising at least one strut and/or a flexible jacket.
